(19) United States
(12) Patent Application Publication
(10) Pub. No.: US 2024/0335582 A1
(43) Pub. Date: Oct. 10, 2024

(54) ATOMIZING AROMA DIFFUSER

(71) Applicant: MMAS LLC, CARY, NC (US)

(72) Inventor: XIAOYUAN MA, Cary, NC (US)

(21) Appl. No.: 18/296,434

(22) Filed: Apr. 6, 2023

(51) Int. Cl. A61L 9/14

(52) U.S. Cl. CPC A61L 9/14; A61L 2209/12; A61L 2209/13; A61L 2209/14

(57) ABSTRACT

An atomizing aroma diffuser, comprising a housing, an oil guiding member, an atomizing sheet, and an essential oil bottle; a mounting cavity is provided inside the housing, and the housing is provided with an oil inlet and a mist outlet for connecting the mounting cavity and ambient; the oil guiding member is arranged in the mounting cavity, and an oil storage channel and a bayonet are provided inside of the oil guiding member; the bayonet is arranged at a first end of the oil storage channel and connected to the oil storage channel and the oil inlet; a second end of the oil storage channel is correspondingly connected to the mist outlet; the atomizing sheet is arranged at the mist outlet and configured to block the oil storage channel; a mouth of the essential oil bottle extends into the oil inlet and is detachably connected to the bayonet.

### TECHNICAL FIELD

The present application belongs to the technical field of aroma diffusers, and in particular relates to a novel atomizing aroma diffuser.

### BACKGROUND

The aroma diffuser is a diffuser that uses an atomization device to decompose the dissolved plant essential oil into a cold mist and emit it into the surrounding air. It has the functions of purifying the air, improving the environment, soothing emotions, and relieving stress.

Existing aroma diffusers generally have a built-in liquid storage chamber for storing essential oils, or directly connect a detachable essential oil bottle; Limited by the volume of the main body of the aroma diffuser, the volume of the liquid storage chamber is small, which will result in a small amount of stored essential oil, which is not conducive to long-term use. Although the external essential oil bottle can solve the problem of less oil storage and is conducive to long-term use, the body of the aroma diffuser needs to be equipped with an essential oil bottle to work. When the essential oil bottle is replaced, the aroma diffuser will inevitably stop working. In addition, the essential oil bottle itself has a certain weight and volume, which is not conducive to the use when the user goes out to carry it.

Therefore, it is necessary to propose a novel atomizing aroma diffuser that can work independently and also can be used with essential oil bottles.

### SUMMARY

The present application provides a novel atomizing aroma diffuser, which can solve the technical problems raised in the background art.

In order to achieve the above object, the present application provides the following technical solutions:

An novel atomizing aroma diffuser comprises a housing, an oil guiding member, an atomizing sheet, and an essential oil bottle; a mounting cavity is provided inside the housing, and the housing is provided with an oil inlet and a mist outlet for connecting the mounting cavity and ambient; the oil guiding member is arranged in the mounting cavity, and an oil storage channel and a bayonet are provided inside of the oil guiding member; the bayonet is arranged at a first end of the oil storage channel and connected to the oil storage channel and the oil inlet; a second end of the oil storage channel is correspondingly connected to the mist outlet; the atomizing sheet is arranged at the mist outlet and configured to block the oil storage channel; a mouth of the essential oil bottle extends into the oil inlet and is detachably connected to the bayonet.

Preferably, the oil inlet and the bayonet are configured to be vertical, the oil storage channel is arranged horizontally, and the mist outlet is configured to be inclined upward relative to the oil storage channel, and the atomizing sheet obliquely blocks the oil storage channel.

Preferably, it further comprises an end cover, the end cover is detachably connected with the oil inlet, so as to close or open the oil inlet.

Preferably, the end cover is made of rubber material, and the end cover is connected with the oil inlet by means of a transition fit.

Preferably, it further comprise sleeves, a sleeve hole and an oil discharge hole are provided inside the sleeve, and the oil discharge hole is connected to the sleeve hole and the ambient; the sleeve can extend into the oil inlet and be detachably connected with the bayonet, the mouth of the essential oil bottle is detachably connected with the sleeve hole; a plurality of sleeves is provided, and the sizes of the sleeve holes on each of the sleeves are different.

Preferably, the mouth of the essential oil bottle is threadedly connected with the sleeve hole

Preferably, an inner wall of the bayonet is provided with a convex strip, and the convex strip and the inner wall of the bayonet enclose to form a limiting groove; the sleeve is rotationally connected with the bayonet, and an outer wall of the sleeve is provided with a protrusion; when the sleeve is placed in the bayonet, the protrusion can be driven to engage into the limiting groove by rotating the sleeve.

Preferably, it further comprises a thimble, the thimble is vertically arranged in the first end of the oil storage channel, and a needle of the thimble extends into the bayonet.

Preferably, the needle of the thimble is configured in a pyramid shape, and a plurality of vertically arranged stays are arranged on an upper circumference of the thimble, and a vertical flow guiding groove is formed between two adjacent stays

Preferably, it further comprises a filter, the filter is detachably arranged on a bottom side of the bayonet to close the first end of the oil storage channel; the filter is provided with a filter groove and a guiding port, and the filter groove is connected to the bayonet and the oil storage channel; the guiding port is slidingly matched with a plurality of stays, so that the filter can move along a length direction of the thimble.

Preferably, it further comprises a battery and a control board electrically connected to the atomizing sheet, the battery and the control board are both arranged in the mounting cavity, and the control board is electrically connected to the battery.

Preferably, a charging port electrically connected to the control board is also arranged on the housing.

Preferably, an LED light strip electrically connected to the control board is arranged on the top side of the housing.

Preferably, the LED strip is arranged in a ring shape and encloses the oil inlet.

The present application provides a novel atomizing aroma diffuser. By arranging an oil storage channel in the oil guiding member, the novel atomizing aroma diffuser can be used alone, so as to reduce the overall weight and volume, and improve portability; By forming a bayonet on the oil guiding member, it is ensured that the essential oil bottle is detachably connected to the housing, so that the novel atomizing aroma diffuser can be used with the essential oil bottle, thereby prolonging the use time; Since the oil storage channel can store essential oils, when replacing the essential oil bottle, the atomizing sheet can also normally contact the essential oil to produce aroma mist, thus ensuring the normal operation of the aroma diffuser. The above-mentioned design makes the aroma diffuser suitable for different usage scenarios.

### BRIEF DESCRIPTION OF DRAWINGS

The accompanying drawings are used to provide a further understanding of the application, and constitute a part of the description, and are used to explain the application together with the embodiments of the application, and do not constitute a limitation to the application. In the accompanying drawings:

FIG. 1 shows the first schematic diagram of the use state of the present application;

FIG. 2 shows the second schematic diagram of the use state of the present application;

FIG. 3 shows the right side view of FIG. 2;

FIG. 4 shows the sectional view A-A of FIG. 3;

FIG. 5 shows an enlarged view at A in FIG. 4;

FIG. 6 shows the exploded schematic view I of the present application;

FIG. 7 shows the exploded schematic diagram II of the present application;

FIG. 8 shows the schematic structural view of the oil guiding member and thimble of the present application;

FIG. 9 shows a top view of FIG. 8;

FIG. 10 shows a schematic structural diagram of the filter of the present application;

FIG. 11 shows a schematic structural diagram of the sleeve of the present application.

Reference signs:
1. Housing, 10. Mounting cavity, 11. Oil inlet, 12 Mist outlet, 1P Charging port, 2. Oil guiding member, 21. Bayonet, 211 Convex strip, 2110. Limiting groove, 22. Oil storage channel, 3. atomizing sheet, 4. Essential oil bottles, 41 Film, 5 End cover, 6. Sleeve, 61. Sleeve hole, 610 Oil discharge hole, 62. Protrusion, 7. Thimble, 71. Stay, 710. Flow guiding grooves, 8. Filter, 80. Filter groove, 81. Guiding port, 9. LED strips, P Battery, K. Control board.

### DETAILED DESCRIPTION

The technical solution in the embodiment of the application will be clearly and completely described below in conjunction with the drawings in the embodiment of the application. Apparently, the described embodiments are only some of the embodiments of this application, not all of them. Based on the embodiments in this application, all other embodiments obtained by persons of ordinary skill in the art without making creative efforts belong to the scope of protection of this application.

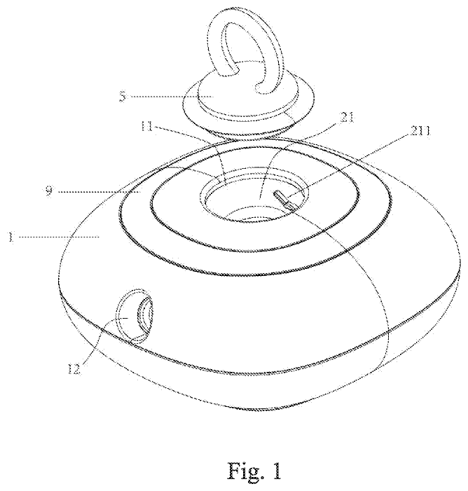

Referring to FIG. 1-7, an atomizing aroma diffuser comprises a housing 1, an oil guiding member 2, an atomizing sheet 3, and an essential oil bottle 4; a mounting cavity 10 is provided inside the housing 1, and a top side of the housing 1 is provided with an oil inlet 11 for connecting the mounting cavity 10 and ambient, the housing 1 is also provided with a mist outlet 12 for connecting the mounting cavity 10 and ambient; the oil guiding member 2 is arranged in the mounting cavity 10, and an oil storage channel 22 and a bayonet 21 are provided inside of the oil guiding member 2; the bayonet 21 is arranged at a first end of the oil storage channel 22 and connected to the oil storage channel 22 and the oil inlet 11; a second end of the oil storage channel 22 is correspondingly connected to the mist outlet 12; the atomizing sheet 3 is arranged at the mist outlet 12 and configured to block the oil storage channel 22; a mouth of the essential oil bottle 4 extends into the oil inlet 11 and is detachably connected to the bayonet 21.

Specifically, the novel atomizing aroma diffuser in this application has two ways of use:

One, as shown in FIG. 1, when used alone, the user directly pours the essential oil from the oil inlet 11 into the bayonet 21 when using it, so that the essential oil flows into the oil storage channel 22 for storage. At this time, the essential oil located at the second end of the oil storage channel 22 is in contact with the atomizing sheet 3, and the atomizing sheet 3 is activated to atomize the contacted essential oil into a fragrance mist and spray it out from the mist discharge port 12. Since the essential oil has fluidity, the essential oil in the oil storage channel 22 will continuously flow and contact the atomizing sheet 3, thereby continuously producing fragrance mist. In order to avoid the excessive volume of the housing 1, the storage space of the oil storage channel 22 is designed to be small, and only a small amount of essential oil can be stored, which is suitable for short-term use.

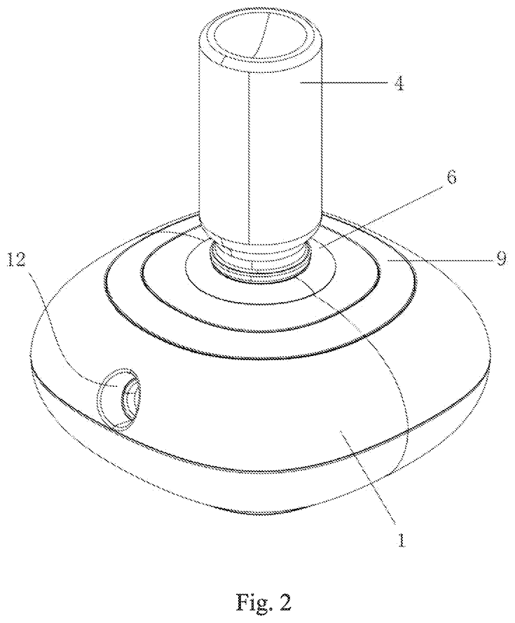

Second, as shown in FIG. 2 to FIG. 5, it is used with the essential oil bottle 4, and the user inserts the mouth of the essential oil bottle 4 with essential oil into the oil inlet 11 and connects with the bayonet 21 when using it, so that the essential oil in the essential oil bottle 4 flow into the oil storage channel 22 for storage; At this time, the essential oil located at the second end of the oil storage channel 22 is in contact with the atomizing sheet 3, and the atomizing sheet 3 is activated to atomize the contacted essential oil into a fragrance mist and spray it out from the mist discharge port 12. Since the volume of the essential oil bottle 4 is larger, more essential oils can be stored, so it is suitable for long-term use.

In daily life, users can choose different methods of use according to different usage scenarios. For example, when going out for use, the aroma diffuser is required to be light as a whole, and the requirement for the amount of essential oil is low, so the first method of use can be used; while for home use, there is no requirement for the overall weight and volume of the aroma diffuser, but the requirement for the amount of essential oil is higher, so the second method of use can be used.

To sum up, in this application, by forming the oil storage channel 22 in the oil guide 2, the novel atomizing aroma diffuser can be used alone, so as to reduce the overall weight and volume, and improve portability; By forming a bayonet 21 on the oil guiding member 2, it is ensured that the essential oil bottle 4 is detachably connected to the housing 1, so that the novel atomizing aroma diffuser can be used with the essential oil bottle 4, thereby prolonging the use time; Since the oil storage channel 22 can store essential oils, when replacing the essential oil bottle 4, the atomizing sheet 3 can also normally contact the essential oil to produce aroma mist, thus ensuring the normal operation of the aroma diffuser. The above-mentioned design makes the aroma diffuser suitable for different usage scenarios.

It should be noted that the atomizing sheet 3 in this application belongs to the prior art, and it can atomize liquid after being powered on. Since it is relatively common in this field, its specific structure and circuit will not be described in detail in this application.

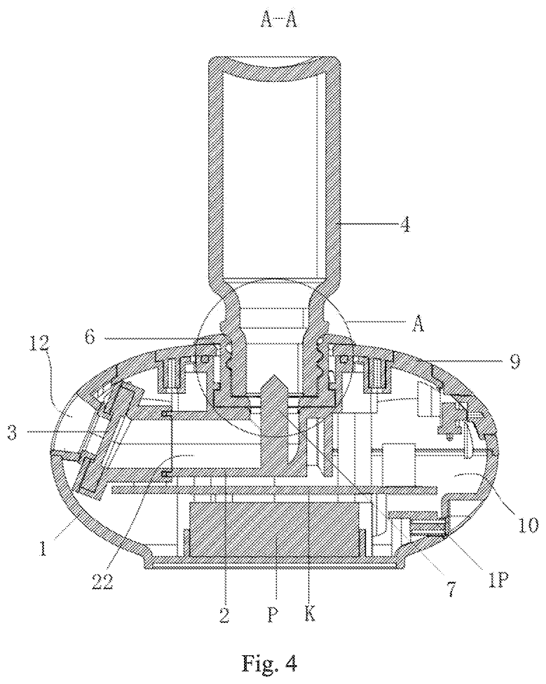

Referring to FIG. 4 to FIG. 7, the oil inlet 11 and the bayonet 21 are configured to be vertical, the oil storage channel 22 is arranged horizontally, and the mist outlet 12 is configured to be inclined upward relative to the oil storage channel 22, and the atomizing sheet 3 obliquely blocks the oil storage channel 22.

Specifically, both the oil inlet 11 and the bayonet 21 are arranged vertically, so that the essential oil bottle 4 can be arranged vertically on the housing 1, so that the essential oil in the essential oil bottle 4 can flow into the liquid storage channel under the action of gravity; The mist outlet 12 is inclined upward relative to the oil storage channel 22, and the atomizing sheet 3 obliquely blocks the oil storage channel 22, so that the fragrance mist can fully contact with the air, volatilize more thoroughly, and avoid the accumulation of fragrance mist and cause table area oil.

On the other hand, the oil storage channel 22 is arranged horizontally, which can ensure that the essential oil in the oil storage channel 22 is always in contact with the atomizing sheet 3. As the remaining amount of essential oil decreases, the essential oil no longer fills the oil storage channel 22, the contact area between the essential oil and the atomizing sheet 3 becomes smaller, and the fragrance mist produced will also decrease, thereby prompting the user that the essential oil is used up.

It should be noted that the oil storage channel 22 in this application can also be arranged downwards, so that the essential oil has a tendency to move down and contact the atomizing sheet 3 under the action of its own gravity, and as the remaining amount of essential oil decreases, the contact area between the essential oil and the atomizing sheet 3 remains unchanged. This design can make the production of fragrance mist consistent until the essential oil is completely consumed.

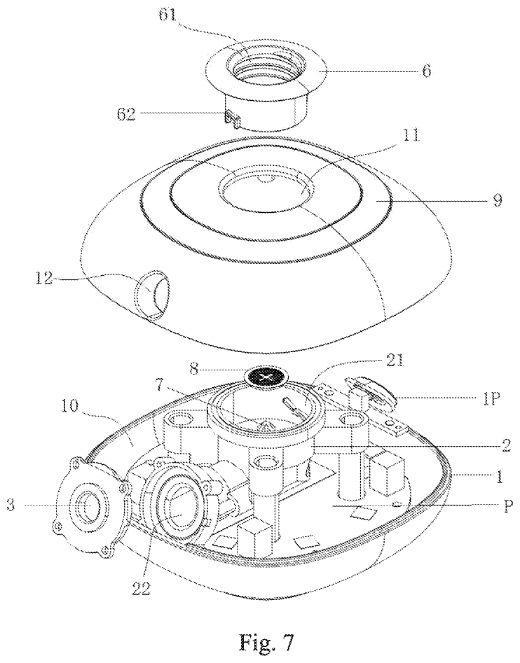

Referring to FIG. 1 and FIG. 7, it further comprises an end cover 5, the end cover 5 is detachably connected to the oil inlet 11, so as to close or open the oil inlet 11.

Specifically, when the novel atomizing aroma diffuser is used alone, the end cover 5 can be used to close the oil inlet 11, so as to prevent external dust from entering the oil storage channel 22 and depositing to block the atomizing sheet 3, and to prevent the essential oil in the oil storage channel 22 from volatilizing and overflowing from the oil inlet 11 to the outside.

There are many ways to detachably connect the end cover 5 and the bayonet 21. The end cover 5 is made of rubber material, and the end cover 5 is connected with the oil inlet 11 through a transition fit. This design can improve the sealing ability of the end cover 5 and reduce the possibility of essential oil overflowing from the oil inlet 11 to the outside.

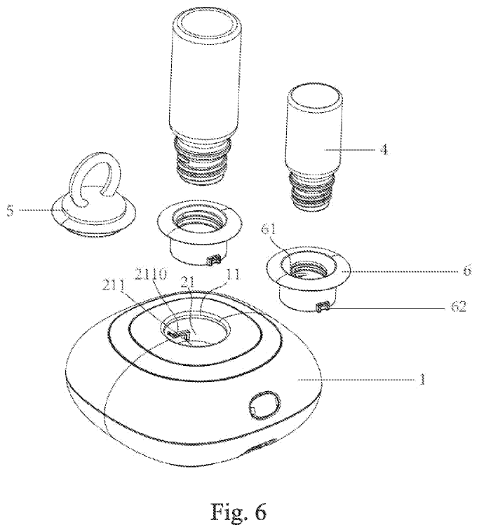

Referring to FIG. 6 to FIG. 7, considering that there are various types of essential oil bottles 4 on the market, and the volumes of various types of essential oil bottles 4 are different, the size of the bottle mouth is also different, in order to make the novel atomizing aroma diffuser in this application can be used with different types of essential oils bottle 4 used, this application also comprises sleeves 6, a sleeve hole 61 and an oil discharge hole 610 are provided inside the sleeve 6, and the oil discharge hole 610 is connected to the sleeve hole 61 and the ambient; the sleeve 6 can extend into the oil inlet 11 and be detachably connected with the bayonet 21, the mouth of the essential oil bottle 4 is detachably connected with the sleeve hole 61; a plurality of sleeves 6 is provided, and the sizes of the sleeve holes 61 on each of the sleeves 6 are different.

Specifically, when installing, the user first selects the sleeve 6 according to the size of the mouth of the essential oil bottle 4, snaps the sleeve hole 61 into the mouth of the essential oil bottle 4, and then inserts the sleeve 6 into the oil inlet 11 and connects the bayonet 21, so that the essential oil bottle 4 and the sleeve 6 are mounted on the housing 1 together. At this time, the essential oil in the essential oil bottle 4 will flow out from the bottle, and enter the oil storage channel 22 through the oil discharge hole 610.

To sum up, the sleeve holes 61 of the sleeves 6 correspond to the designs of the mouths of various types of essential oil bottles 4, which can improve the adaptability of the novel atomizing aroma diffuser.

It should be noted that, since the sizes of the sleeve holes 61 on each sleeve 6 are different, each sleeve 6 corresponds to one type of essential oil bottle 4. According to the size of the mouth of various essential oil bottles 4 on the market, manufacturers can produce the sleeves 6 with same shape but different sizes of the sleeve holes 61 for users to choose.

Referring FIG. 6, furthermore, there are many ways to connect the mouth of the essential oil bottle 4 and the sleeve hole 61, and common connection mode is threaded connection. In this application, the internal thread is formed in the sleeve hole 61, and an external thread that mates with an internal thread is formed on the sleeve 6.

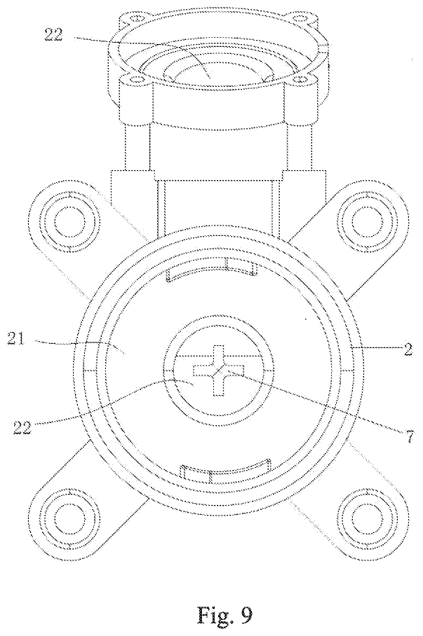

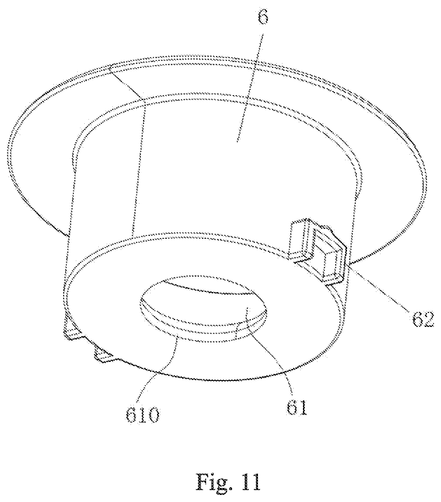

Referring FIG. 7 to FIG. 11, furthermore, there are many ways to connect the sleeve 6 and the bayonet 21, such as clamping, screwing, etc., which are not limited in this application. For the convenience of understanding, in this application, an inner wall of the bayonet 21 is provided with a convex strip 211, and the convex strip 211 and the inner wall of the bayonet 21 enclose to form a limiting groove 2110; the sleeve 6 is rotationally connected with the bayonet 21, and an outer wall of the sleeve 6 is provided with a protrusion 62; when the sleeve 6 is placed in the bayonet 21, the protrusion 62 can be driven to engage into the limiting groove 2110 by rotating the sleeve 6. Similarly, the protrusion 62 can be driven out of the limiting groove 2110 by rotating the sleeve 6 in the opposite direction, and then the sleeve 6 can be taken out from the bayonet 21 by moving the essential oil bottle 4 upwards.

Referring FIG. 4 to FIG. 11, some essential oil bottles 4 currently on the market are sealed with a film 41. Before installation, the film 41 needs to be torn to expose the mouth of the bottle, which causes the essential oil to easily overflow the essential oil bottle 4 and be sprinkled on the housing 1 during the installation of the essential oil bottle 4, resulting in poor user experience. To solve this problem, the present application also comprises a thimble 7, which is vertically installed in the first end of the oil storage channel 22, and the needle of the thimble 7 extends into the bayonet 21.

Specifically, when mounting the essential oil bottle 4, the user does not need to tear the film 41. When the mouth of the essential oil bottle 4 enters the bayonet 21, the needle of the thimble 7 will pierce the film 41 into the essential oil bottle 4, so that the liquid in the essential oil bottle 4 will flow out and flow into the oil storage channel 22. When changing the essential oil bottle 4, the mouth of the essential oil bottle 4 is separated from the bayonet 21, and the essential oil bottle 4 can be driven to break away from the thimble 7. Since the process of mounting and replacing the essential oil bottle 4 is carried out in the bayonet 21, it is possible to prevent the essential oil from spilling on the housing 1 and improve user experience.

It should be noted that if it is used in combination with the sleeve 6, during the connection between the sleeve 6 and the bayonet 21, the thimble 7 will pass through the oil discharge hole 610 and pierce the film 41 at the mouth of the bottle. In addition, the diameter of the thimble 7 needs to be smaller than the diameter of the connection opening between the bayonet 21 and the oil storage channel 22, so that the thimble 7 will not block the connection opening between the bayonet 21 and the oil storage channel 22, when the novel atomizing aroma diffuser is used alone, to ensure that the essential oil poured into the bayonet 21 from the oil inlet 11 can also smoothly enter the storage channel.

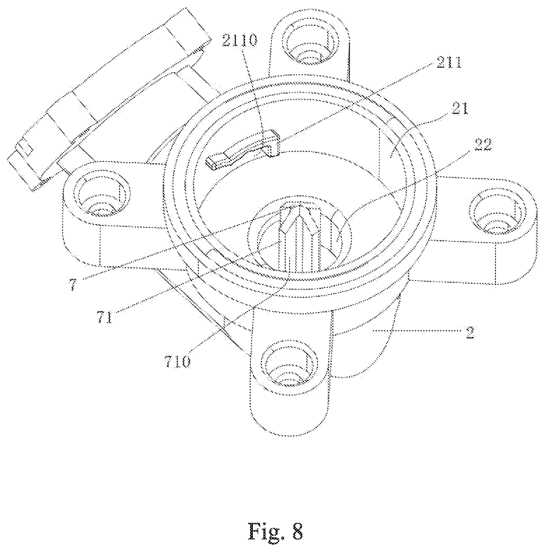

Referring to FIG. 8, the needle of the thimble 7 is configured in a pyramid shape, and a plurality of vertically stays 71 are arranged in an array on the upper circumference of the thimble 7, and vertical flow guiding grooves 710 are formed between any two adjacent stays 71.

Specifically, the needle of the thimble 7 is configured in a pyramid shape, which can improve the puncture ability of the thimble 7; and the plurality of stays 71 can expand the puncture range of the thimble 7, so that the needle of the thimble 7 pierces the film 41 and then stretches the puncture hole. In addition, the vertical flow guiding grooves 710 formed between the plurality of stays 71 have a flow guiding function, and can accurately guide the essential oil in the essential oil bottle 4 into the oil storage channel 22, which reduces the possibility of oil remaining on the edge of the bottom side of the bayonet 21 and the mouth of the bottle.

It should also be noted that due to the design of the flow guide groove 710, the bayonet 21 can also be connected to the oil storage channel 22 through the flow guiding grooves 710, so even if the diameter of the thimble 7 is equal to the connection opening between the bayonet 21 and the oil storage channel 22, the essential oil poured into the bayonet 21 from the oil inlet 11 can smoothly enter the storage channel through the flow guiding grooves 710.

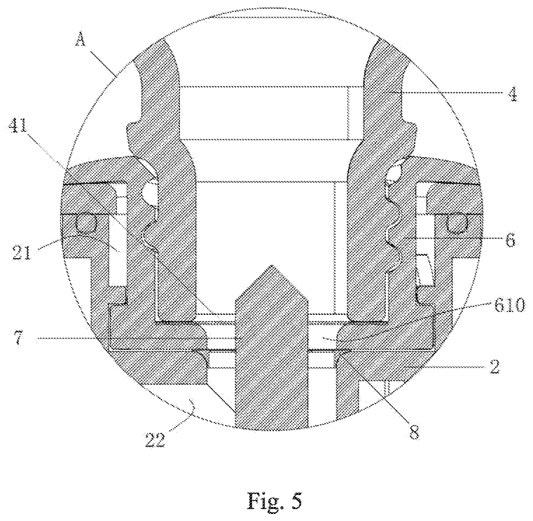

Referring to FIGS. 5 to 10, during use, external sundries or impurities of the essential oil itself may accumulate in the oil storage channel 22, and after long-term use, the oil storage channel 22 and the atomizing sheet 3 will be blocked, which leads to an unsatisfactory atomization effect, and even various essential oil residues mixed to make the fragrance impure. To solve this problem, the present application also comprises a filter 8, which is detachably arranged on the bottom side of the bayonet 21 to cover the first end of the oil storage channel 22. The filter 8 is provided with filter grooves 80 and a guiding port 81, the filter grooves 80 are connected to the bayonet 21 and the oil storage channel 22. The guiding port 81 is slidingly matched with the plurality of stays 71, so that the filter 8 can move along the length direction of the thimble 7.

Specifically, when installing the filter 8, place the filter 8 above the thimble 7, and make the guide port 81 correspond to a plurality of stays 71, and then move the filter 8 down along the length direction of the thimble 7, so that the filter 8 can block the connection opening between the bayonet 21 and the oil storage channel 22.

When the novel atomizing aroma diffuser is used alone, the essential oil is directly poured into the bayonet 21 from the oil inlet 11, and the essential oil enters the oil storage channel 22 through the filter groove 80 on the filter 8, and the impurities in the essential oil and foreign matters will be screened out by the filter 8. When the novel atomizing aroma diffuser is used in combination with the essential oil bottle 4, the mouth of the essential oil bottle 4 is connected to the bayonet 21, and the essential oil flows out from the essential oil bottle 4, and enters the oil storage channel 22 through the filter grooves 80, and the impurities in the essential oil will be screened out by the filter 8.

Through the cooperation of the plurality of stays 71 and the guiding port 81, the rotation of the filter 8 can be prevented, and the rollover of the filter 8 caused by the impact of the essential oil can be avoided.

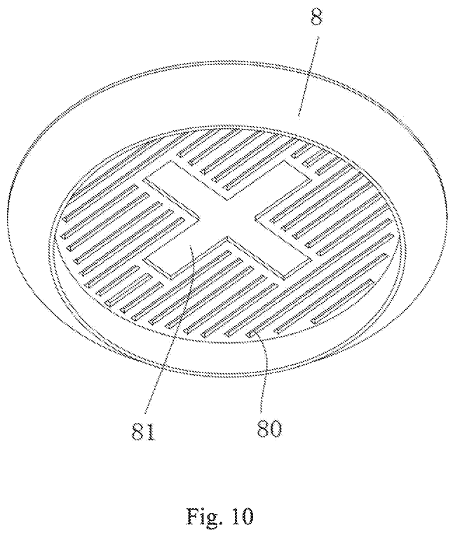

Referring to FIGS. 8 and 10, further, in order to prevent the filter 8 from moving up and down arbitrarily, in this application, the edge of the connection opening between the bayonet 21 and the oil storage channel 22 can be rounded, and the peripheral edge of the filter 8 is also provided with a corresponding rounded groove. Through the mutual cooperation between the two, the filter 8 can be installed on the bottom side of the bayonet 21 more stably, and the filter 8 can be prevented from moving up and down at will.

In addition, when the novel atomizing aroma diffuser is used with the sleeve 6, the sleeve 6 is connected to the bayonet 21, so that the bottom side of the sleeve 6 touches the edge of the filter screen, and then the filter screen is fixed, and the essential oil flows out from the essential oil bottle 4, and passes through the oil discharge hole 610 and the filter grooves 80 into the oil storage channel 22.

It should be noted that in order to avoid clogging of the filter 8, the filter 8 needs to be disassembled and cleaned regularly. When disassembling, the filter can be hooked with the help of a tool, or the housing 1 can be turned upside down directly, so that the filter moves along the thimble 7 toward the oil inlet 11 and falls out.

Referring to FIGS. 4 and 7, the application also comprises a battery P and a control board K electrically connected to the atomizing sheet 3, the battery P and the control board K are both arranged in the mounting cavity 10, and the control board K is electrically connected to the battery P.

Specifically, the battery P is used to supply power to the control board K and the atomizing sheet 3, and the control board K is used to control the operating state of the atomizing sheet 3; wherein, a switch electrically connected to the control board K can be provided on the housing 1, for the user to press to startup or shutdown the atomizing sheet 3; or directly arrange a Bluetooth module on the control panel K, and send instructions to the control panel K through the mobile phone to control the startup and shutdown of the atomizing sheet 3.

Since the relevant technology is relatively existing and is a common technical means in this field, the specific structure and circuit thereof will not be described in detail in this application.

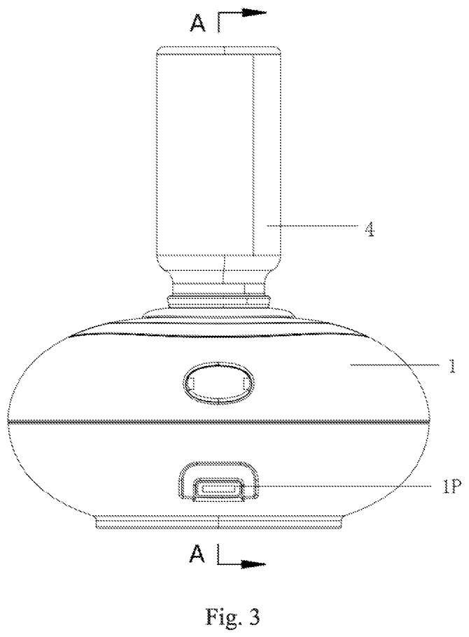

Referring to FIG. 3, FIG. 4 and FIG. 7, further, the battery P can be a detachable battery P, or a rechargeable accumulator, preferably a accumulator in this application. A charging port 1P electrically connected to the control board K is mounted on the housing 1 to facilitate charging for the user.

Referring to FIG. 4 and FIG. 7, an LED light strip 9 electrically connected to the control board K is installed on the top side of the housing 1.

The LED light strip 9 has a circular design and encloses the oil inlet 11. This design can make the novel atomizing aroma diffuser more aesthetically pleasing; and it is convenient for users to replace the essential oil at night.

In addition, a switch electrically connected to the control board K can be installed on the housing 1 for the user to press to startup or shutdown the LED light strip 9; Or directly arrange a Bluetooth module on the control board K, and send instructions to the control board K through the mobile phone to control to startup or shutdown of the LED light strip 9.

It should also be noted that although the embodiments of the present application have been shown and described, those skilled in the art can understand that many modifications, changes, substitutions and variations can be made to these embodiments without departing from the principle and spirit of the present application.

What is claimed is:

1. An atomizing aroma diffuser, comprising:
a housing (1), a mounting cavity (10) is provided inside the housing (1), an oil inlet (11) connecting the mounting cavity (10) and ambient is provided on a top side of the housing (1), the housing (1) is also provided with a mist outlet (12) connecting the mounting cavity (10) and the ambient;
an oil guiding member (2), the oil guiding member (2) is arranged in the mounting cavity (10), and a bayonet (21) and an oil storage channel (22) are provided inside the oil guiding member (2); the bayonet (21) is arranged at a first end of the oil storage channel (22) and connected to the oil storage channel (22) and the oil inlet (11); a second end of the oil storage channel (22) is correspondingly connected to the mist outlet (12);
an atomizing sheet (3), the atomizing sheet (3) is arranged at the mist outlet (12) and configured to block the oil storage channel (22);
an essential oil bottle (4), a mouth of the essential oil bottle (4) extends into the oil inlet (11) and is detachably connected to the bayonet (21).

2. The atomizing aroma diffuser according to claim 1, wherein the oil inlet (11) and the bayonet (21) are configured to be vertical, the oil storage channel (22) is arranged horizontally, and the mist outlet (12) is configured to be inclined upward relative to the oil storage channel (22), and the atomizing sheet (3) obliquely blocks the oil storage channel (22).

3. The atomizing aroma diffuser according to claim 1, wherein further comprising an end cover (5), the end cover (5) is detachably connected with the oil inlet (11), so as to close or open the oil inlet (11).

4. The atomizing aroma diffuser according to claim 1, wherein further comprising sleeves (6), a sleeve hole (61) and an oil discharge hole (610) are provided inside the sleeve (6), and the oil discharge hole (610) is connected to the sleeve hole (61) and the ambient; the sleeve (6) can extend into the oil inlet (11) and be detachably connected with the bayonet (21), the mouth of the essential oil bottle (4) is detachably connected with the sleeve hole (61); a plurality of sleeves (6) is provided, and the sizes of the sleeve holes (61) on each of the sleeves (6) are different.

5. The atomizing aroma diffuser according to claim 4, wherein the mouth of the essential oil bottle (4) is threadedly connected with the sleeve hole (61); an inner wall of the bayonet (21) is provided with a convex strip (211), and the convex strip (211) and the inner wall of the bayonet (21) enclose to form a limiting groove (2110); the sleeve (6) is rotationally connected with the bayonet (21), and an outer wall of the sleeve (6) is provided with a protrusion (62); when the sleeve (6) is placed in the bayonet (21), the protrusion (62) can be driven to engage into the limiting groove (2110) by rotating the sleeve (6).

6. The atomizing aroma diffuser according to claim 1, wherein further comprising a thimble (7), the thimble (7) is vertically arranged in the first end of the oil storage channel (22), and a needle of the thimble (7) extends into the bayonet (21).

7. The atomizing aroma diffuser according to claim 6, wherein the needle of the thimble (7) is configured in a pyramid shape, and a plurality of vertically arranged stays (71) are arranged on an upper circumference of the thimble (7), and a vertical flow guiding groove (710) is formed between two adjacent stays (71).

8. The atomizing aroma diffuser according to claim 7, wherein further comprising a filter (8), the filter (8) is detachably arranged on a bottom side of the bayonet (21) to close the first end of the oil storage channel (22); the filter (8) is provided with a filter groove (80) and a guiding port (81), and the filter groove (80) is connected to the bayonet (21) and the oil storage channel (22); the guiding port (81) is slidingly matched with a plurality of stays (71), so that the filter (8) can move along a length direction of the thimble (7).

9. The atomizing aroma diffuser according to claim 1, wherein further comprising a battery (P) and a control board (K) electrically connected to the atomizing sheet (3), the battery (P) and the control board (K) are both arranged in the mounting cavity (10), and the control board (K) is electrically connected to the battery (P).

10. The atomizing aroma diffuser according to claim 9, wherein an LED light strip (9) electrically connected to the control board (K) is arranged on the top side of the housing (1).
